(19) United States
(12) Patent Application Publication
(10) Pub. No.: US 2003/0229371 A1
(43) Pub. Date: Dec. 11, 2003

(54) Offset surgical scissors

(76) Inventor: Warren A. Whitworth, Midlothian, VA (US)

Correspondence Address: John H. Thomas, P.C., 1561 East Main Street, Richmond, VA, 23219, US

(21) Appl. No.: 10/166,457

(22) Filed: Jun. 10, 2002

(51) Int. Cl. A61B 17/32

(52) U.S. Cl. 606/174

(57) ABSTRACT

A surgical scissors includes a shaft having a pair of rings on one end and a pair of tips on the opposite end. The scissors further have an offset portion located in the rings half of the shaft so that a user has an unobstructed view down the shaft to the tips during use.

The present invention relates to surgical devices and specifically to an offset surgical scissors apparatus.

### BACKGROUND OF THE INVENTION

Medical professionals perform a wide range of procedures requiring specialized equipment. Much of the equipment is designed to make surgery safer and less traumatic to a patient's body. As these medical and surgical methods evolve, so do the performance requirements for the equipment that facilitates that work.

Currently, in common saphenectomy procedures, for instance, a vein in a patient's leg is harvested for different types of bypass surgeries. During minimally invasive saphenectomy procedures. An incision is made in the patient's thigh and the vein is isolated. Then scissors are inserted into the incision and a tunnel is created up an down the patient's leg to a point approximately 15 cms proximal and distal to the initial incision to safely isolate and harvest the vein. Medical professionals look down the shaft of the scissors to see the tips and guide cutting accordingly in this procedure. With conventional straight scissors, as well as with Metzenbaum scissors having slightly curved tips, a user has difficulty looking down the shaft of the scissors, because the user's fingers in the rings of the scissors block the view. The user has to move him or herself and/or the scissors around to get a clear view of the tips. This movement of the professional and the scissors is inconvenient, is more intrusive inside the patient, and can place limitations on the procedure.

Shears and scissors having bent handles are known. But the problem with known scissors is that the bent or offset portions are relatively close to a scissors tips along the length of the shaft of the scissors. Known offset locations towards the tip ends of scissors require more space for operation in a patient making the procedure more intrusive by definition. Particularly with scissors having a long shaft, it is preferred to irritate as little surrounding tissue as possible in a patient during use. There is a need to improve scissors so that a user is able to have a better sight line down the shaft and of the tips of the scissors and still be as least intrusive as possible inside a patient.

### SUMMARY OF THE INVENTION

Accordingly, it is an object of the present invention to provide an improved surgical scissors that will facilitate the use of the scissors during a medical procedure. Further, those scissors should be as minimally intrusive as possible.

In a preferred embodiment, a surgical scissors comprises a shaft comprising a pair of opposing sections connected at a hinge. A pair of rings defines one end of the shaft, and a pair of tips defines the opposite end. The shaft has a tips half and a rings half, each half making up a half of the length of the shaft. This shaft further comprises an offset portion located in the rings half of the shaft whereby a user of the scissors has an unobstructed view down the shaft to the tips during use. The offset portion may comprised two bends. The shaft may further comprise a rear portion from the rings to the offset portion and a front portion from the tips to the offset portion, and the near and far portions are substantially parallel to each other. The lines defined by the rear portion and the far portion may be substantially straight lines that are spaced approximately one inch apart. The length from the tips to the offset portion may be about half the overall length of the scissors. Each bend may be approximately forty-five degrees with each in the opposite direction to the other. Still further alternatively, the length of the shaft and the rings to the offset portion may be at least two inches.

### BRIEF DESCRIPTION OF THE DRAWINGS

FIG. 1 is a perspective view of a pair of offset surgical scissors in accordance with the present invention with the scissors in the closed position.

FIG. 1A is a perspective view of a pair of offset surgical scissors in accordance with the present invention with the offset surgical scissors in the open position.

FIG. 2 is a top plan view of the offset surgical scissors shown in FIG. 1.

FIG. 3 is side elevation view of the offset surgical scissors in accordance with the present invention as also seen in the other figures.

### DETAILED DESCRIPTION

The present invention is a surgical scissors having a shaft with an offset portion in it. Specifically, the offset portion is located in the same half of the scissors as the rings, in other words, the half of the scissors opposite the working tips of the scissors. The discussion herein is primarily in the context of a commercial embodiment of a pair of scissors in accordance with the present invention sold by Genzyme Corporation under the name “Offset Surgical Scissors”. The invention is particularly useful in connection with saphenectomy procedures, but the scissors could also be used in any procedure where surgical scissors are used and especially where the user is required to look down the shaft during use. The commercial embodiments of the scissors are nine inch and eleven inch models. But of course other sizes and variations are possible within the spirit of this invention.

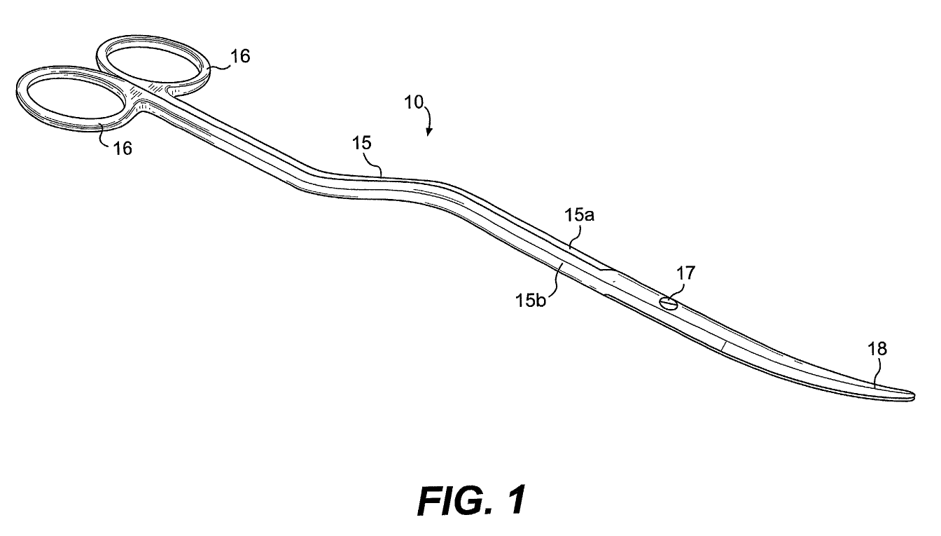

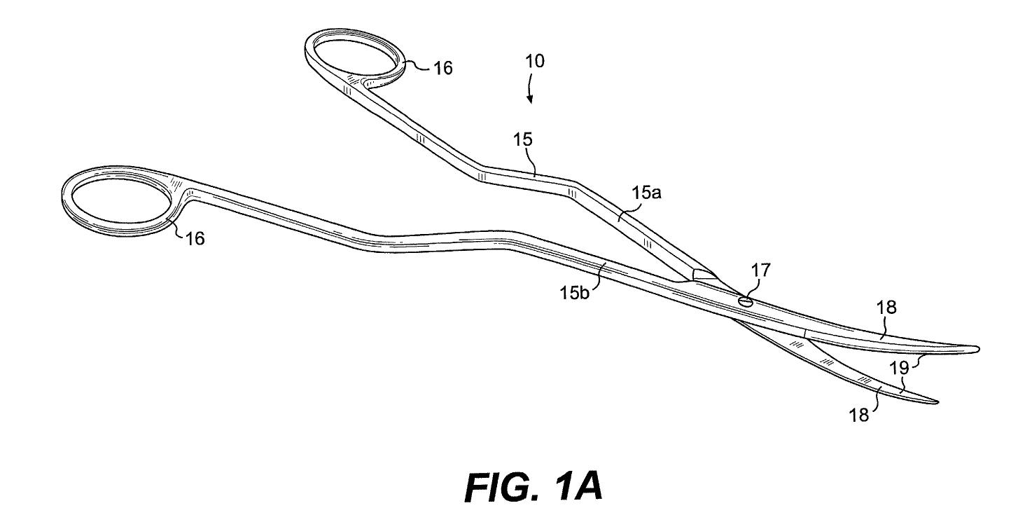

Referring now to FIGS. 1 to 3, there is shown scissors 10. Shaft 15 is made up of a pair of opposing sections 15a and 15b that, in the closed position, are straight. Rings 16 make up one end of the shaft 15. Tips 18 make up the opposite end of the shaft 15. The hinge 17 and rings 16 are conventional in size and construction. The shaft sections 15a and 15b are rotatably connected to each other by hinge 17.

The tips 18 include cutting surface 19. As shown, the tips 18 and cutting surface 19 have a curve similar to known types of surgical scissors commonly referred to as Metzenbaum scissors. Metzenbaum scissors generically include shaft portions that rotate about a hinge. The shaft, excluding the curved tips, are rotated about the hinge in substantially a common plane. The curve of the tips extends out of that plane. In the scissors 10 shown, the tips 18 curve upwardly from the plane of scissors rotation—upwardly being defined as the opposite side of the plane from the side by which a users fingers will engage and work with the scissors.

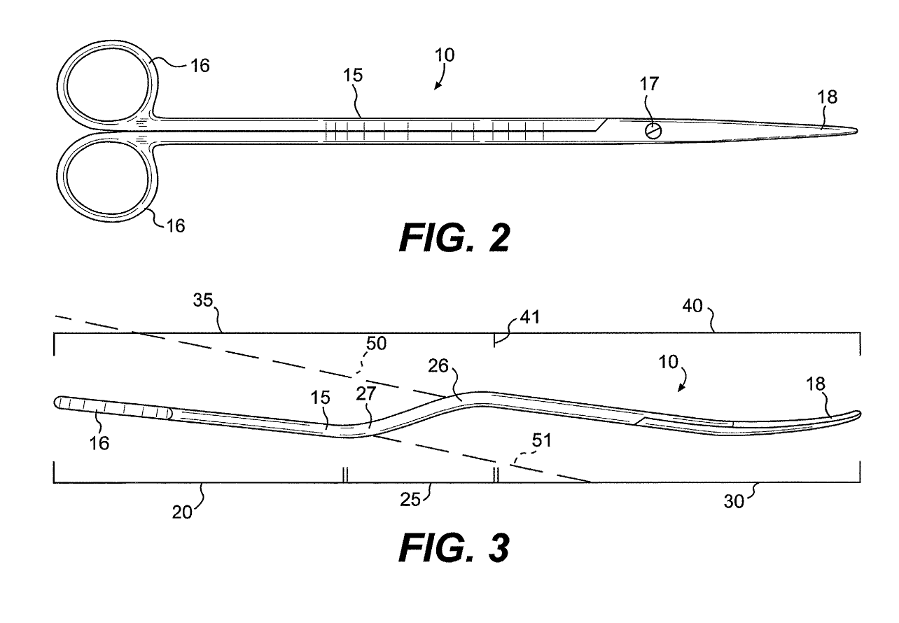

Referring now specifically to FIG. 3, shaft 15 has multiple portions, specifically a near portion 20, an offset portion 25, and a far portion 30. The near portion 20 of the shaft 15 includes rings 16 and extends from the end of the rings up to the offset portion 25. Preferably, the near portion 20 is made up of the rings 16 and approximately two inches of the shaft 15 so that a user may guide and steady the scissors 10 with a finger other than the fingers in the rings. Alternatively, the length of the shaft 15 of the near portion 20 between the rings 16 and the offset portion 25 can be shorter or longer depending on the preference of the user and the requirements of a procedure. The near portion 20 of the shaft 15 is, as can be seen in the side view, a straight line. In other words, the rings 16 and remaining shaft of the near portion 20 are coplanar and remain coplanar as the scissors 10 are operated.

The far portion 30 of the shaft 15 includes tips 18, the hinge 17, and a length of shaft 15 up to the offset portion 25. The far portion 30 is linear generally in a straight line except for the slight curved portion of the tips 18 as seen in the example in the drawings.

The offset portion 25 includes a pair of bends 26 and 27 such that the near portion 20 and far portion 30 are substantially parallel to each other. In the preferred embodiment shown in the drawings, the angle of bend in the bends 26 and 27 are each approximately 45 degrees. Other angles are possible, but gradual angles in the range of 20 degrees to 70 degrees are preferred. The angles may be reciprocal so that the near portion 20 and far portion 30 are parallel. Alternatively, the angles of the bends 26 and 27 could be different. If the angles are different, then the angles must be selected so that the rings 16, and the fingers of a user in those rings, will not block the line of sight 50 shown in dotted lines down the far portion of the shaft 30. In a preferred embodiment of a nine inch length pair of scissors, the offset distance between the line defined by the near portion 51 and the line of sight defined by the far portion 50 is approximately one inch.

The near bend 27 is part of the offset portion 25 adjacent the near portion 20. The far bend 26 is part of the offset portion 25 adjacent the far portion 30. The offset 25 is placed in the half of the length of the scissors 35 that includes the rings 16. In other words, the far bend 26 is in the rings half 35 of the length of the scissors but, as shown in the drawings, begins substantially at the mid point 41 of the length of the pair of scissors 10. In this way, the tips half 40 of the scissors are generally straight but for the upward curvature of the tips 18 typical in Metzenbaum scissors.

The visual limitations of a procedure such as a saphenectomy mean that the tips are only visible inside a patient's body for about five or six inches (12-15 cm) from where the incision is made and the scissors are inserted. The offset portion 25, because it is in the rings half 35 of the shaft 15, is behind or at the end of the maximum visibility of a medical professional during use of the scissors. If the offset portion 25 were in the tips half 40 of the shaft 15, then the insertion of the scissors into a patient would be more intrusive by having to allow for the insertion of the offset portion 25 completely inside a patient. The placement of the offset portion 25 in the rings half 35, in combination with the gradual angle of the offset portion (approximately 45 degrees in the embodiment shown) means that it is possible for a user to work with the scissors to the maximum practical visual limits of the procedure.

The scissors 10 shown are preferably constructed of stainless steel. Any material is possible as long as it can be adequately sterilized if it is to be reused.

While the invention has been described with reference to specific embodiments thereof, it will be understood that numerous variations, modifications and additional embodiments are possible, and all such variations, modifications, and embodiments are to be regarded as being within the spirit and scope of the invention.

What is claimed is:

1. A surgical scissors comprising
a shaft comprising a pair of opposing sections connected at a hinge,
a pair of rings defining one end of the shaft and a pair of tips defining the opposite end, the shaft having a tips half and a rings half, each half making up a half of the length of the shaft, and
wherein the shaft further comprises an offset portion located in the rings half of the shaft,
whereby a user has an unobstructed view down the shaft to the tips during use.

2. A surgical scissors as described in claim 1, wherein the offset portion comprises two bends.

3. A surgical scissors as described in claim 1, wherein the shaft further comprises a near portion from the rings to the offset portion and a far portion from the tips to the offset portion, and the near and far portions are substantially parallel to each other.

4. A surgical scissors as described in claim 3, wherein the lines defined by the near portion and the far portion are substantially straight lines and are spaced approximately one inch apart.

5. A surgical scissors as described in claim 1, wherein the length from the tips to the offset portion is about half the overall length of scissors.

6. A surgical scissors as described in claim 3, wherein the length of the far portion is approximately half the overall length of the scissor.

7. A surgical scissors as described in claim 2, wherein each bend is approximately 45° with each bend being in the opposite direction to the other.

8. A surgical scissors as described in claim 1, wherein the length of the shaft from the rings to the offset portion is at least two inches.
